(19) United States
(12) Patent Application Publication
(10) Pub. No.: US 2023/0192901 A1
(43) Pub. Date: Jun. 22, 2023

(54) INDUCTION OF CONCURRENT PULMONARY IMMUNE MODULATION AND REGENERATION BY PROTEIN MEDIATED CONJUGATION OF IMMUNE REGULATORY CELLS WITH ENDOGENOUS PROGENITOR CELLS

(71) Applicant: Therapeutic Solutions International, Inc., Oceanside, CA (US)

(72) Inventors: Thomas E. Ichim, Oceanside, CA (US); Famela Ramos, Oceanside, CA (US); James Veltmeyer, Oceanside, CA (US); Timothy G. Dixon, Oceanside, CA (US)

(73) Assignee: Therapeutic Solutions International, Inc., Oceanside, CA (US)

(21) Appl. No.: 17/989,588

(22) Filed: Nov. 17, 2022

Related U.S. Application Data
(60) Provisional application No. 63/274,341, filed on Nov. 1, 2021.

(51) Int. Cl. C07K 16/46; A61P 31/14

(52) U.S. Cl. CPC C07K 16/468; A61P 31/14; C07K 2317/22

(57) ABSTRACT

Disclosed are means, methods and compositions of matter useful for treatment of inflammatory pulmonary diseases such as COVID-19 through administration of agents that facilitate interaction between immune modulatory cells and endogenous pulmonary progenitor cells. In one embodiment a bispecific antibody capable of facilitating the interaction between CD25 on T regulatory cells and CD47 on pulmonary epithelial stem cells is described.

### CROSS-REFERENCE TO RELATED APPLICATIONS

This application claims priority to U.S. Provisional Application Ser. No. 63/274,341, titled “Induction of Concurrent Pulmonary Immune Modulation and Regeneration by Protein Mediated Conjugation of Immune Regulatory Cells with Endogenous Progenitor Cells”, filed Nov. 1, 2022, which is hereby incorporated by reference in its entirety.

### FIELD OF THE INVENTION

The invention pertains to the field of immune modulation, more specifically it pertains to the utilization of “facilitatory proteins” to enable cellular communications between immune regulatory and regenerative cells.

### BACKGROUND OF THE INVENTION

SARS-CoV-2 is an enveloped, positive-sense, single-stranded RNA virus of the subgenus Sarbecovirus which belongs to the genus Betacoronavirus [1, 2]. The main strains of this family are 229E (alpha coronavirus), NL63 (alpha coronavirus), 0C43 (beta coronavirus), and HKU1 (beta coronavirus), which are relatively innocuous and cause the common cold, as well as more virulent strains such as MERS-CoV (the beta coronavirus that causes Middle East Respiratory Syndrome, or MERS) [3-10]. Rapidly after its identification, scientists found that SARS-CoV-2 possesses 88% identity to two bat-derived severe acute respiratory syndrome (SARS)-like coronaviruses, bat-SL-CoVZC45 and bat-SL-CoVZXC21 which were collected in 2018 in Zhoushan, eastern China. It was also found that SARS-CoV-2 has 79% homology to SARS-CoV and 50% homology to MERS-CoV [11]. Pathology of COVID-19 is associated with pulmonary damage caused by localized leukocytic infiltration resulting in lung fluid leakage and reduced respiratory capacity. Negative association between neutrophil infiltration, inflammatory markers in blood, and in BAL exists with patient survival [12]. Suppression of inflammation using various means such as antibodies to IL-6 have shown clinical benefit in some situations [13-15].

Unfortunately, there is still a great need to develop approaches that selectively inhibit inflammation, when needed, without causing systemic consequences. Additionally, to date, tissue specific targeting of pulmonary inflammation has not been performed using monoclonal antibody-based approaches to our knowledge. T regulatory cells are immune regulators characterized by expression of the transcription factor FoxP3 and ability to suppress various arms of the immune response such as T cell proliferation and cytokine production, dendritic cell maturation and neutrophil activation [16]. The fundamental role of T regulatory cells in immune homeostasis is witnessed in animal lacking expression of FoxP3 who die as a result of a systemic autoimmune condition. Further support is findings that humans possessing a mutation in FoxP3 also suffer from polyautoimmunity [17].

Additionally, it has been shown that T regulatory cells possess the ability to induce tissue regeneration in some model systems, in part through direct secretion of growth factors such as VEGF, FGF-1, or FGF-2, or through the indirect stimulation of by acting muscle cells and/or fibroblast cells in various local tissue environments [18].

### SUMMARY

Preferred embodiments are directed to methods of treating covid-19 or associated sequelae comprising administration of a protein, wherein said protein is capable of facilitating interaction between an immune regulatory cell and a pulmonary epithelial progenitor cell.

Preferred embodiments are directed to embodiments wherein said protein is a cameloid antibody.

Preferred embodiments are directed to embodiments wherein said protein is a bispecific antibody.

Preferred embodiments are directed to embodiments wherein said protein microbody.

Preferred embodiments are directed to embodiments wherein said protein is a chimeric protein.

Preferred embodiments are directed to embodiments wherein said covid-19 associated sequelae is post-covid pulmonary fibrosis.

Preferred embodiments are directed to embodiments wherein said immune regulatory cell is a NKT cell.

Preferred embodiments are directed to embodiments wherein said immune regulatory cell is a Th2 cell.

Preferred embodiments are directed to embodiments wherein said immune regulatory cell is a Th3 cell.

Preferred embodiments are directed to embodiments wherein said immune regulatory cell is a T regulatory cell.

Preferred embodiments are directed to embodiments wherein said T regulatory cell expresses membrane bound TGF-beta.

Preferred embodiments are directed to embodiments wherein said T regulatory cell expresses FoxP3.

Preferred embodiments are directed to embodiments wherein said T regulatory cell is capable of stimulating angiogenesis.

Preferred embodiments are directed to embodiments wherein said T regulatory cell is capable of increasing activity of a pulmonary progenitor cell.

Preferred embodiments are directed to embodiments wherein said pulmonary progenitor cell is a type 2 pulmonary epithelial cell.

Preferred embodiments are directed to embodiments wherein said pulmonary progenitor cell expresses CD133.

Preferred embodiments are directed to embodiments wherein said bispecific antibody is capable of binding CD25 on one arm and CD47 on another arm.

Preferred embodiments are directed to embodiments wherein said bispecific antibody is capable of binding CD25 on one arm and CD133 on another arm.

Preferred embodiments are directed to embodiments wherein said bispecific antibody is administered by aerosol.

Preferred embodiments are directed to embodiments wherein said bispecific antibody is administered by aerosol.

### BRIEF DESCRIPTION OF THE DRAWINGS

FIG. 1 is a bar graph showing dose-dependent suppression of pulmonary inflammation in mice from an antibody treatment.

### DETAILED DESCRIPTION OF THE INVENTION

The invention provides, intra-alia, bispecific antibodies facilitating interaction between immune regulatory and regenerative cells. In one embodiment antibodies capable of binding both CD25 and CD47 are disclosed. Other combinations including antibodies that bind surface TGF-beta and CD47, TGF-beta and CD133, TGF-beta and CD34 and TGF-beta and VEGF-receptors.

In further aspects, the invention provides heterodimeric antibodies comprising: a) a first heavy chain comprising a first Fc domain, an optional domain linker and a first antigen binding domain comprising an scFv that binds a first antigen; b) a second heavy chain comprising a heavy chain comprising a heavy chain constant domain comprising a second Fc domain, a hinge domain, a CH1 domain and a variable heavy domain; and c) a light chain comprising a variable light domain and a light chain constant domain; wherein said variable heavy domain and said variable light domain form a second antigen binding domain that binds a second antigen, wherein one of said first and second antigen binding domains binds human CD25 and the other binds human CD47. In an additional aspect, the invention provides heterodimeric bispecific antibodies comprising: a) a first heavy chain comprising: i) a first variant Fc domain; and ii) a single chain Fv region (scFv) that binds a first antigen, wherein said scFv region comprises a first variable heavy chain, a variable light chain and a charged scFv linker, wherein said charged scFv linker covalently attaches said first variable heavy chain and said variable light chain; and b) a second heavy chain comprising a VH-CH1-hinge-CH2-CH3 monomer, wherein VH is a second variable heavy chain and CH2-CH3 is a second variant Fc domain; and c) a light chain; wherein said second variant Fc domain comprises amino acid substitutions.

Thus, the present invention provides bispecific immunomodulatory antibodies. An ongoing problem in antibody technologies is the desire for “bispecific” antibodies that bind to two (or more) different antigens simultaneously, in general thus allowing the different antigens to be brought into proximity and resulting in new functionalities and new therapies. In general, these antibodies are made by including genes for each heavy and light chain into the host cells (generally, in the present invention, genes for two heavy chain monomers and a light chain as outlined herein). This generally results in the formation of the desired heterodimer (A-B), as well as the two homodimers (A-A and B-B). However, a major obstacle in the formation of bispecific antibodies is the difficulty in purifying the heterodimeric antibodies away from the homodimeric antibodies and/or biasing the formation of the heterodimer over the formation of the homodimers. To solve this issue, there are a number of mechanisms that can be used to generate the heterodimers of the present invention. In addition, as will be appreciated by those in the art, these mechanisms can be combined to ensure high heterodimerization. Thus, amino acid variants that lead to the production of heterodimeric antibodies are referred to as “heterodimerization variants”. As discussed below, heterodimerization variants can include steric variants (e.g. the “knobs and holes” or “skew” variants described below and the “charge pairs” variants described below) as well as “pI variants”, which allows purification of homodimers away from heterodimers.

One mechanism is generally referred to in the art as “knobs and holes” (“KIH”) or sometimes herein as “skew” variants, referring to amino acid engineering that creates steric influences to favor heterodimeric formation and disfavor homodimeric formation can also optionally be used; this is sometimes referred to as “knobs and holes”; as described in Ridgway et al., Protein Engineering 9(7):617 (1996); Atwell et al., J. Mol. Biol. 1997 270:26; U.S. Pat. No. 8,216,805, US 2012/0149876, all of which are hereby incorporated by reference in their entirety. The Figures identify a number of “monomer A-monomer B” pairs that include “knobs and holes” amino acid substitutions. In addition, as described in Merchant et al., Nature Biotech. 16:677 (1998), these “knobs and hole” mutations can be combined with disulfide bonds to skew formation to heterodimerization. Of use in the present invention are T366S/L368A/Y407V paired with T366W, as well as this variant with a bridging disulfide, T366S/L368A/Y407V/Y349C paired with T366W/S354C, particularly in combination with other heterodimerization variants including pI variants as outlined below. An additional mechanism that finds use in the generation of heterodimeric antibodies is sometimes referred to as “electrostatic steering” or “charge pairs” as described in Gunasekaran et al., J. Biol. Chem. 285(25):19637 (2010), hereby incorporated by reference in its entirety. This is sometimes referred to herein as “charge pairs”. In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “steric variants”. These include, but are not limited to, D221E/P228E/L368E paired with D221R/P228R/K409R (e.g. these are “monomer corresponding sets) and C220E/P228E/368E paired with C220R/E224R/P228R/K409R and others shown in the Figures.

In the present invention, in some embodiments, pI variants are used to alter the pI of one or both of the monomers and thus allowing the isoelectric purification of A-A, A-B and B-B dimeric proteins. In the present invention, there are several basic mechanisms that can lead to ease of purifying heterodimeric proteins; one relies on the use of pI variants, such that each monomer has a different pI, thus allowing the isoelectric purification of A-A, A-B and B-B dimeric proteins. Alternatively, some scaffold formats, such as the “triple F” or “bottle opener” format, also allows separation on the basis of size. As is further outlined below, it is also possible to “skew” the formation of heterodimers over homodimers. Thus, a combination of steric heterodimerization variants and pI or charge pair variants find particular use in the invention. Additionally, as more fully outlined below, scaffolds that utilize scFv(s) such as the Triple F format can include charged scFv linkers (either positive or negative), that give a further pI boost for purification purposes. As will be appreciated by those in the art, some Triple F formats are useful with just charged scFv linkers and no additional pI adjustments, although the invention does provide the use of skew variants with charged scFv linkers as well (and combinations of Fc, FcRn and KO variants discussed herein). In the present invention that utilizes pI as a separation mechanism to allow the purification of heterodimeric proteins, amino acid variants can be introduced into one or both of the monomer polypeptides; that is, the pI of one of the monomers (referred to herein for simplicity as “monomer A”) can be engineered away from monomer B, or both monomer A and B change be changed, with the pI of monomer A increasing and the pI of monomer B decreasing. As is outlined more fully below, the pI changes of either or both monomers can be done by removing or adding a charged residue (e.g. a neutral amino acid is replaced by a positively or negatively charged amino acid residue, e.g. glycine to glutamic acid), changing a charged residue from positive or negative to the opposite charge (aspartic acid to lysine) or changing a charged residue to a neutral residue (e.g. loss of a charge; lysine to serine). A number of these variants are shown in the Figures. In addition, suitable pI variants for use in the creation of heterodimeric antibodies herein are those that are isotypic, e.g. importing pI variants from different IgG isotypes such that pI is changed without introducing significant immunogenicity; see FIG. 29 from US Publication No. 20140288275, hereby incorporated by reference in its entirety.

Accordingly, in this embodiment of the present invention provides for creating a sufficient change in pI in at least one of the monomers such that heterodimers can be separated from homodimers. As will be appreciated by those in the art, and as discussed further below, this can be done by using a “wild type” heavy chain constant region and a variant region that has been engineered to either increase or decrease its pI (wt A−+B or wt A−−B), or by increasing one region and decreasing the other region (A+−B− or A− B+). In general, a component of some embodiments of the present invention are amino acid variants in the constant regions of antibodies that are directed to altering the isoelectric point (pI) of at least one, if not both, of the monomers of a dimeric protein to form “pI heterodimers” (when the protein is an antibody, these are referred to as “pI antibodies”) by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the present invention. As will be appreciated by those in the art, the number of pI variants to be included on each or both monomer(s) to get good separation will depend in part on the starting pI of the scFv and Fab of interest. That is, to determine which monomer to engineer or in which “direction” (e.g. more positive or more negative), the Fv sequences of the two target antigens are calculated and a decision is made from there. As is known in the art, different Fvs will have different starting pIs which are exploited in the present invention. In general, as outlined herein, the pIs are engineered to result in a total pI difference of each monomer of at least about 0.1 logs, with 0.2 to 0.5 being preferred as outlined herein. Furthermore, as will be appreciated by those in the art and outlined herein, in some cases (depending on the format) heterodimers can be separated from homodimers on the basis of size (e.g. Molecular weight). For example, as shown in some embodiments, some formats result in homodimers and heterodimers with different sizes (e.g. for bottle openers, one homodimer is a “dual scFv” format, one homodimer is a standard antibody, and the heterodimer has one Fab and one scFv).

### EXAMPLES

One of the major pathologies of COVID-19 is the opening of the endothelial gap junctions allowing for fluid to enter the extracapillary spaces. This results in fluid accumulating in the alveoli, which makes breathing compromised, even in some cases during mechanical ventilation.

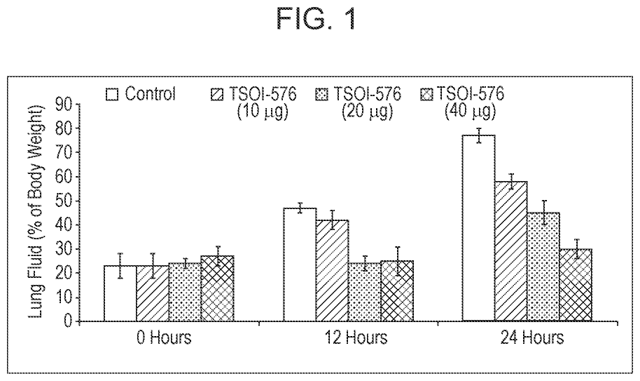

The TSOI-576 antibody was administered intravenously into BALB/c mice treated with 100 nanograms of endotoxin per mouse. Endotoxin functions by binding to toll like receptor 4 and causes a pathology that has been reported to be similar to COVID-19 induced ARDS. The lung weight of the animal was measured at time points of zero, 12 hours and 24 hours. Ten mice per group where assessed for the experiment. As can be seen, a significant dose-dependent suppression of pulmonary inflammation was seen as resulted of the antibody treatment. Results are shown in FIG. 1. Additionally, examination of the lungs revealed TSOI-576 also resulted in dose-dependent suppression of neutrophil infiltration, interleukin-6 content in the lung and overall suppression of fibrosis in mice allowed to live 4 weeks and assessed for fibrotic tissue content by hydroxyproline staining.

### REFERENCES

1. Pal, M., et al., Severe Acute Respiratory Syndrome Coronavirus-2 (SARS-CoV-2): An Update. Cureus, 2020. 12(3): p. e7423.
2. Malik, Y. A., Properties of Coronavirus and SARS-CoV-2. Malays J Pathol, 2020. 42(1): p. 3-11.
3. Huang, P., et al., Nucleic acid visualization assay for Middle East Respiratory Syndrome Coronavirus (MERS-CoV) by targeting the UpE and N gene. PLoS Negl Trop Dis, 2021. 15(3): p. e0009227.
4. Rabets, A., et al., The Potential of Developing Pan-Coronaviral Antibodies to Spike Peptides in Convalescent COVID-19 Patients. Arch Immunol Ther Exp (Warsz), 2021. 69(1): p. 5.
5. Alshehri, M. A., et al., On the Prevalence and Potential Functionality of an Intrinsic Disorder in the MERS-CoV Proteome. Viruses, 2021. 13(2).
6. Park, B. K., et al., MERS-CoV and SARS-CoV-2 replication can be inhibited by targeting the interaction between the viral spike protein and the nucleocapsid protein. Theranostics, 2021. 11(8): p. 3853-3867.
7. Chen, J., et al., Development of A MERS-CoV Replicon Cell Line for Antiviral Screening. Virol Sin, 2021. 36(4): p. 730-735.
8. Abdelghany, T. M., et al., SARS-CoV-2, the other face to SARS-CoV and MERS-CoV. Future predictions. Biomed J, 2021. 44(1): p. 86-93.
9. Ansariniya, H., et al., Comparison of Immune Response between SARS, MERS, and COVID-19 Infection, Perspective on Vaccine Design and Development. Biomed Res Int, 2021. 2021: p. 8870425.
10. Saha, J., et al., A comparative genomics-based study of positive strand RNA viruses emphasizing on SARS-CoV-2 utilizing dinucleotide signature, codon usage and codon context analyses. Gene Rep, 2021. 23: p. 101055.
11. Lu, R., et al., Genomic characterisation and epidemiology of 2019 novel coronavirus: implications for virus origins and receptor binding. Lancet, 2020. 395(10224): p. 565-574.
12. Zhang, C., et al., A Novel Scoring System for Prediction of Disease Severity in COVID-19. Front Cell Infect Microbiol, 2020. 10: p. 318.
13. Liu, B., et al., Can we use interleukin-6 (IL-6) blockade for coronavirus disease 2019 (COVID-19)-induced cytokine release syndrome (CRS)? J Autoimmun, 2020. 111: p. 102452.
14. Salama, C., et al., Tocilizumab in Patients Hospitalized with Covid-19 Pneumonia. N Engl J Med, 2021. 384(1): p. 20-30.
15. Zhang, C., et al., Cytokine release syndrome in severe COVID-19: interleukin-6 receptor antagonist tocilizumab may be the key to reduce mortality. Int J Antimicrob Agents, 2020. 55(5): p. 105954.
16. Oberg, H. H., et al., Regulatory Interactions Between Neutrophils, Tumor Cells and T Cells. Front Immunol, 2019. 10: p. 1690.
17. Bacchetta, R., F. Barzaghi, and M. G. Roncarolo, From IPEX syndrome to FOXP3 mutation: a lesson on immune dysregulation. Ann N Y Acad Sci, 2018. 1417(1): p. 5-22.
18. Schiaffino, S., et al., Regulatory T cells and skeletal muscle regeneration. FEBS J, 2017. 284(4): p. 517-524.

1. A method of treating covid-19 or associated sequelae comprising administration of a protein, wherein said protein is capable of facilitating interaction between an immune regulatory cell and a pulmonary epithelial progenitor cell.

2. The method of claim 1, wherein said protein is a cameloid antibody.

3. The method of claim 1, wherein said protein is a bispecific antibody.

4. The method of claim 1, wherein said protein microbody.

5. The method of claim 1, wherein said protein is a chimeric protein.

6. The method of claim 1, wherein said covid-19 associated sequelae is post-covid pulmonary fibrosis.

7. The method of claim 1, wherein said immune regulatory cell is a NKT cell.

8. The method of claim 1, wherein said immune regulatory cell is a Th2 cell.

9. The method of claim 1, wherein said immune regulatory cell is a Th3 cell.

10. The method of claim 1, wherein said immune regulatory cell is a T regulatory cell.

11. The method of claim 10, wherein said T regulatory cell expresses membrane bound TGF-beta.

12. The method of claim 10, wherein said T regulatory cell expresses FoxP3.

13. The method of claim 10, wherein said T regulatory cell is capable of stimulating angiogenesis.

14. The method of claim 10, wherein said T regulatory cell is capable of increasing activity of a pulmonary progenitor cell.

15. The method of claim 14, wherein said pulmonary progenitor cell is a type 2 pulmonary epithelial cell.

16. The method of claim 10, wherein said pulmonary progenitor cell expresses CD133.

17. The method of claim 3, wherein said bispecific antibody is capable of binding CD25 on one arm and CD47 on another arm.

18. The method of claim 3, wherein said bispecific antibody is capable of binding CD25 on one arm and CD133 on another arm.

19. The method of claim 17, wherein said bispecific antibody is administered by aerosol.

20. The method of claim 18, wherein said bispecific antibody is administered by aerosol.
